SEQUENCE LISTING (9 sequences)

SEQ ID NO: 1 - RNA, 2208 nt, Homo sapiens
ucuuuggcuuuuuuuggcggagcuggggcgcccuccggaagcguuuccaacuuuccagaaguuucucgggacgggcaggaggggguggggacugccauauauagaucccgggagcaggggagcgggcuaagaguagaaucgugucgcggcucgagagcgagagucacgucccggcgcuagcccagcccgacccaggcccaccguggugcacgcaaaccacuuccuggccaugcgcucccuccugcuucucagcgccuucugccuccuggaggcggcccuggccgccgaggugaagaaaccugcagccgcagcagcuccuggcacugcggagaaguugagccccaaggcggccacgcuugccgagcgcagcgccggccuggccuucagcuuguaccaggccauggccaaggaccaggcaguggagaacauccuggugucacccguggugguggccucgucgcuagggcucgugucgcugggcggcaaggcgaccacggcgucgcaggccaaggcagugcugagcgccgagcagcugcgcgacgaggaggugcacgccggccugggcgagcugcugcgcucacucagcaacuccacggcgcgcaacgugaccuggaagcugggcagccgacuguacggacccagcucagugagcuucgcugaugacuucgugcgcagcagcaagcagcacuacaacugcgagcacuccaagaucaacuuccgcgacaagcgcagcgcgcugcaguccaucaacgagugggccgcgcagaccaccgacggcaagcugcccgaggucaccaaggacguggagcgcacggacggcgcccugcuagucaacgccauguucuucaagccacacugggaugagaaauuccaccacaagaugguggacaaccguggcuucauggugacucgguccuauaccgugggugucaugaugaugcaccggacaggccucuacaacuacuacgacgacgagaaggaaaagcugcaaaucguggagaugccccuggcccacaagcucuccagccucaucauccucaugccccaucacguggagccucucgagcgccuugaaaagcugcuaaccaaagagcagcugaagaucuggauggggaagaugcagaagaaggcuguugccaucuccuugcccaagggugugguggaggugacccaugaccugcagaaacaccuggcugggcugggccugacugaggccauugacaagaacaaggccgacuugucacgcaugucaggcaagaaggaccuguaccuggccagcguguuccacgccaccgccuuugaguuggacacagauggcaaccccuuugaccaggacaucuacgggcgcgaggagcugcgcagccccaagcuguucuacgccgaccaccccuucaucuuccuagugcgggacacccaaagcggcucccugcuauucauugggcgccugguccggccuaagggugacaagaugcgagacgaguuauagggccucagggugcacacaggauggcaggaggcauccaaaggcuccugagacacaugggugcuauugggguugggggggaggugagguaccagccuuggauacuccaugggguggggguggaaaaacagaccgggguucccgugugccugagcggaccuucccagcuagaauucacuccacuuggacaugggccccagauaccaugaugcugagcccggaaacuccacauccugugggaccugggccauagucauucugccugcccugaaagucccagaucaagccugccucaaucaguauucauauuuauagccagguaccuucucaccugugagaccaaauugagcuaggggggucagccagcccucuucugacacuaaaacaccucagcugccuccccagcucuaucccaaccucucccaacuauaaaacuaggugcugcagccccugggaccaggcacccccagaaugaccuggccgcagugaggcggauugagaaggagcucccaggaggggcuucugggcagacucuggucaagaagcaucgugucuggcguuguggggaugaacuuuuuguuuuguuucuuccuuuuuuaguucuucaaagauagggagggaagggggaacaugagccuuuguugcuaucaauccaagaacuuauuuguacauuuuuuuuuucaauaaaacuuuuccaaugacauuuuguuggagcguggaaaaaa

SEQ ID NO: 2 - DNA, 21 bp, Homo sapiens
cuacgacgacgagaaggaatt

SEQ ID NO: 3 - DNA, 21 bp, Homo sapiens
uuccuucucgucgucguagta

SEQ ID NO: 4 - DNA, 21 bp, Homo sapiens
agcccucuucugacacuaatt

SEQ ID NO: 5 - DNA, 21 bp, Homo sapiens
uuagugucagaagagggcugg

SEQ ID NO: 6 - DNA, 21 bp, Homo sapiens
ggacaggccucuacaacuatt

SEQ ID NO: 7 - DNA, 21 bp, Homo sapiens
uaguuguagaggccugucctt

SEQ ID NO: 8 - DNA, 23 bp, Artificial Sequence
  HSP47 gRNA
ccgactgtacggacccagctcag

SEQ ID NO: 9 - DNA, 4140 bp, Artificial Sequence
  Cas9
atggacaagaagtactccattgggctcgatatcggcacaaacagcgtcggctgggccgtcattacggacgagtacaaggtgccgagcaaaaaattcaaagttctgggcaataccgatcgccacagcataaagaagaacctcattggcgccctcctgttcgactccggggagacggccgaagccacgcggctcaaaagaacagcacggcgcagatatacccgcagaaagaatcggatctgctacctgcaggagatctttagtaatgagatggctaaggtggatgactctttcttccataggctggaggagtcctttttggtggaggaggataaaaagcacgagcgccacccaatctttggcaatatcgtggacgaggtggcgtaccatgaaaagtacccaaccatatatcatctgaggaagaagcttgtagacagtactgataaggctgacttgcggttgatctatctcgcgctggcgcatatgatcaaatttcggggacacttcctcatcgagggggacctgaacccagacaacagcgatgtcgacaaactctttatccaactggttcagacttacaatcagcttttcgaagagaacccgatcaacgcatccggagttgacgccaaagcaatcctgagcgctaggctgtccaaatcccggcggctcgaaaacctcatcgcacagctccctggggagaagaagaacggcctgtttggtaatcttatcgccctgtcactcgggctgacccccaactttaaatctaacttcgacctggccgaagatgccaagcttcaactgagcaaagacacctacgatgatgatctcgacaatctgctggcccagatcggcgaccagtacgcagacctttttttggcggcaaagaacctgtcagacgccattctgctgagtgatattctgcgagtgaacacggagatcaccaaagctccgctgagcgctagtatgatcaagcgctatgatgagcaccaccaagacttgactttgctgaaggcccttgtcagacagcaactgcctgagaagtacaaggaaattttcttcgatcagtctaaaaatggctacgccggatacattgacggcggagcaagccaggaggaattttacaaatttattaagcccatcttggaaaaaatggacggcaccgaggagctgctggtaaagcttaacagagaagatctgttgcgcaaacagcgcactttcgacaatggaagcatcccccaccagattcacctgggcgaactgcacgctatcctcaggcggcaagaggatttctacccctttttgaaagataacagggaaaagattgagaaaatcctcacatttcggataccctactatgtaggccccctcgcccggggaaattccagattcgcgtggatgactcgcaaatcagaagagaccatcactccctggaacttcgaggaagtcgtggataagggggcctctgcccagtccttcatcgaaaggatgactaactttgataaaaatctgcctaacgaaaaggtgcttcctaaacactctctgctgtacgagtacttcacagtttataacgagctcaccaaggtcaaatacgtcacagaagggatgagaaagccagcattcctgtctggagagcagaagaaagctatcgtggacctcctcttcaagacgaaccggaaagttaccgtgaaacagctcaaagaagactatttcaaaaagattgaatgtttcgactctgttgaaatcagcggagtggaggatcgcttcaacgcatccctgggaacgtatcacgatctcctgaaaatcattaaagacaaggacttcctggacaatgaggagaacgaggacattcttgaggacattgtcctcacccttacgttgtttgaagatagggagatgattgaagaacgcttgaaaacttacgctcatctcttcgacgacaaagtcatgaaacagctcaagaggcgccgatatacaggatgggggcggctgtcaagaaaactgatcaatgggatccgagacaagcagagtggaaagacaatcctggattttcttaagtccgatggatttgccaaccggaacttcatgcagttgatccatgatgactctctcacctttaaggaggacatccagaaagcacaagtttctggccagggggacagtcttcacgagcacatcgctaatcttgcaggtagcccagctatcaaaaagggaatactgcagaccgttaaggtcgtggatgaactcgtcaaagtaatgggaaggcataagcccgagaatatcgttatcgagatggcccgagagaaccaaactacccagaagggacagaagaacagtagggaaaggatgaagaggattgaagagggtataaaagaactggggtcccaaatccttaaggaacacccagttgaaaacacccagcttcagaatgagaagctctacctgtactacctgcagaacggcagggacatgtacgtggatcaggaactggacatcaatcggctctccgactacgacgtggatcatatcgtgccccagtcttttctcaaagatgattctattgataataaagtgttgacaagatccgataaaaatagagggaagagtgataacgtcccctcagaagaagttgtcaagaaaatgaaaaattattggcggcagctgctgaacgccaaactgatcacacaacggaagttcgataatctgactaaggctgaacgaggtggcctgtctgagttggataaagccggcttcatcaaaaggcagcttgttgagacacgccagatcaccaagcacgtggcccaaattctcgattcacgcatgaacaccaagtacgatgaaaatgacaaactgattcgagaggtgaaagttattactctgaagtctaagctggtctcagatttcagaaaggactttcagttttataaggtgagagagatcaacaattaccaccatgcgcatgatgcctacctgaatgcagtggtaggcactgcacttatcaaaaaatatcccaagcttgaatctgaatttgtttacggagactataaagtgtacgatgttaggaaaatgatcgcaaagtctgagcaggaaataggcaaggccaccgctaagtacttcttttacagcaatattatgaattttttcaagaccgagattacactggccaatggagagattcggaagcgaccacttatcgaaacaaacggagaaacaggagaaatcgtgtgggacaagggtagggatttcgcgacagtccggaaggtcctgtccatgccgcaggtgaacatcgttaaaaagaccgaagtacagaccggaggcttctccaaggaaagtatcctcccgaaaaggaacagcgacaagctgatcgcacgcaaaaaagattgggaccccaagaaatacggcggattcgattctcctacagtcgcttacagtgtactggttgtggccaaagtggagaaagggaagtctaaaaaactcaaaagcgtcaaggaactgctgggcatcacaatcatggagcgatcaagcttcgaaaaaaaccccatcgactttctcgaggcgaaaggatataaagaggtcaaaaaagacctcatcattaagcttcccaagtactctctctttgagcttgaaaacggccggaaacgaatgctcgctagtgcgggcgagctgcagaaaggtaacgagctggcactgccctctaaatacgttaatttcttgtatctggccagccactatgaaaagctcaaagggtctcccgaagataatgagcagaagcagctgttcgtggaacaacacaaacactaccttgatgagatcatcgagcaaataagcgaattctccaaaagagtgatcctcgccgacgctaacctcgataaggtgctttctgcttacaataagcacagggataagcccatcagggagcaggcagaaaacattatccacttgtttactctgaccaacttgggcgcgcctgcagccttcaagtacttcgacaccaccatagacagaaagcggtacacctctacaaaggaggtcctggacgccacactgattcatcagtcaattacggggctctatgaaacaagaatcgacctctctcagctcggtggagacagcagggctgaccccaagaagaagaggaaggtgtga

(19) United States
(12) Patent Application Publication
(10) Pub. No.: US 2021/0260095 A1
(43) Pub. Date: Aug. 26, 2021

(54) MEDICINE USING HSP47 INHIBITOR TO ENHANCE SENSITIVITY TO CHEMOTHERAPEUTIC AGENT

(71) Applicant: NITTO DENKO CORPORATION, Osaka (JP)

(72) Inventors: Akihiro Yoneda, Sapporo-shi (JP); Yasuaki Tamura, Sapporo-shi (JP); Norio Takei, Sapporo-shi (JP); Kaori Sawada, Sapporo-shi (JP)

(21) Appl. No.: 17/269,479

(22) PCT Filed: Aug. 21, 2019
(86) PCT No.: PCT/JP2019/032609

§ 371 Date: Feb. 18, 2021

(30) Foreign Application Priority Data
Aug. 22, 2018 (JP) 2018-155147

(51) Int. Cl. A61K 31/7105; A61K 31/513; A61K 31/713; A61K 33/243; A61K 45/06; A61P 35/00

(52) U.S. Cl. CPC A61K 31/7105; A61K 31/513; A61P 35/00; A61K 33/243; A61K 45/06; A61K 31/713

(57) ABSTRACT

Provided is a medicine containing a HSP47 inhibitor to increase sensitivity of a cancer patient to a chemotherapeutic agent.

### TECHNICAL FIELD

The present invention relates to a medicine and a method for enhancing the sensitivity of a cancer patient to a chemotherapeutic agent.

### BACKGROUND ART

Regarding therapeutic methods for cancer, surgical therapy, radiation therapy, particle beam therapy, chemotherapy, molecular targeted therapy, and the like are available. Among these, treatment by means of chemotherapeutic agents has taken an important position in cancer treatment; however, there are cancers having resistance to chemotherapeutic agents, and side effects may occur when large amounts of a chemotherapeutic agent are administered.

With regard to cancer treatment, combination therapies in which one or more kinds of chemotherapeutic agents having different advantages in combination are also implemented. In combination therapies for cancer, for example, cisplatin and 5-fluorouracil or the like are used. However, there are still numerous cancers that are difficult to treat with chemotherapeutic agents, and there is a demand for new treatment methods.

In Patent Document 1, a method for treating a malignant tumor using a molecule that targets HSP47 is described; however, it is not described whether a molecule targeting HSP47 can increase the sensitivity of a cancer patient to a chemotherapeutic agent.

### CITATION LIST

### Patent Document 1: U.S. Patent Publication No. 2017/0218365

### SUMMARY OF THE INVENTION

### Problems to be Solved by the Invention

An object of the invention is to enhance the sensitivity of a cancer patient to a chemotherapeutic agent.

### Means for Solving the Problems

The inventors of the invention repeatedly conducted a thorough investigation in order to address the above-described problems, and they found that in a case where a molecule targeting HSP47 and cisplatin or 5-fluorouracil are administered, proliferation of various cancer cells can be conspicuously suppressed, that is, the molecule targeting HSP47 can enhance the sensitivity of a cancer patient to a chemotherapeutic agent, as compared to the case of administering the molecule targeting HSP47 alone, or as compared to the case of administering cisplatin or 5-fluorouracil alone, thus completing the invention. Chemotherapeutic agents having actions such as DNA synthesis inhibition and DNA damage can induce cell death by inducing endoplasmic reticulum stress. An HSP47 inhibitor is expected to inhibit the effect exerted by HSP47 for alleviating endoplasmic reticulum stress and to increase the intracellular endoplasmic reticulum stress.

That is, the invention relates to the following.

(1) A medicine for increasing sensitivity of a cancer patient to a chemotherapeutic agent, comprising an HSP47 inhibitor.

(2) The medicine according to (1), wherein the HSP47 inhibitor is an interfering nucleic acid against HSP47, a ribozyme, an antisense nucleic acid, a microRNA, a short hairpin RNA, a vector expressing any of these, or a cell transformed with any of these.

(3) The medicine according to (2), wherein the interfering nucleic acid against HSP47 is a siNA or a siRNA.

(4) The medicine according to any one of (1) to (3), wherein the cancer patient is a patient with breast cancer, colorectal cancer, colon cancer, or pancreatic cancer.

(5) The medicine according to any one of (1) to (4), wherein the chemotherapeutic agent is an alkylating agent, an antimetabolite, an antitumor antibiotic substance, an alkaloid, a hormonal therapeutic agent, a platinum complex, an angiogenesis inhibitory agent, a topoisomerase inhibitory agent, or a microtubule agonist.

(6) The medicine according to any one of (1) to (5), wherein the chemotherapeutic agent is cisplatin (CDDP) or 5-fluorouracil (5-FU).

(7) A medicine for treating cancer, comprising an HSP47 inhibitor and a chemotherapeutic agent.

(8) Use of an HSP47 inhibitor in production of a medicine for the treatment of cancer, the treatment being a treatment of administering a chemotherapeutic agent and an HSP47 inhibitor in combination.

(9) A composition comprising an HSP47 inhibitor for use in the treatment of cancer, the treatment being a treatment of administering a chemotherapeutic agent and an HSP47 inhibitor in combination.

(10) A method for increasing sensitivity of a cancer patient to a chemotherapeutic agent, comprising administering an effective amount of an HSP47 inhibitor to a cancer patient.

(11) The method according to (10), further comprising a step of identifying a patient in need of treatment using a medicine comprising an HSP47 inhibitor.

(12) The method according to (11), wherein the step is a step of quantitatively determining the HSP47 expression in a tumor tissue isolated from a cancer patient.

(13) A method for treating cancer, comprising administering effective amounts of an HSP47 inhibitor and a chemotherapeutic agent to a cancer patient.

(14) A method for treating cancer cells in vitro, comprising treating cancer patient-derived cancer cells in vitro using effective amounts of an HSP47 inhibitor and a chemotherapeutic agent.

### Advantageous Effects of the Invention

The medicine according to the invention can enhance the sensitivity of various cancer patients to a chemotherapeutic agent by inhibiting the expression of HSP47 (including the expression of both or either of HSP47 mRNA and protein) in cancer cells.

Through the combination therapy of the invention, proliferation of various cancer cells can be efficiently suppressed, and cancer can be treated or cured.

According to the invention, the amount of administration of a chemotherapeutic agent can be reduced to a large extent in cancer treatment, and anticancer treatment can be carried out while reducing the risk of side effects caused by the chemotherapeutic agent.

### BRIEF DESCRIPTION OF THE DRAWINGS

FIG. 1 shows the viability of human cancer cells with HSP47 silenced, after treating the human cancer cells with cisplatin (CDDP). MIA-PaCa-2 cells and MDA-MB-231 cells were transfected with control siRNA (siControl, 10 nM) and HSP47 siRNA (siHSP47, 10 nM) in any one of three batches (A, B, and C) and were cultured for 48 hours. After 48 hours from the transfection with siRNA, the cells were treated with cisplatin (CDDP) at a concentration of 0, 1, 5, 10, 20, or 50 μM and were cultured for 24 hours. After 24 hours from the treatment with CDDP, the viability of the cells was determined by a Dye-exclusion assay. The axis of ordinate represents the cell viability (%), and the axis of abscissa represents the concentration (μM) of cisplatin.

FIG. 2 shows the viability of HSP47 knockout human cancer cells treated with CDDP. Human cancer cells (Mock) and HSP47 knockout (KO) cancer cells were treated with cisplatin (CDDP) at a concentration of 0, 1, 5, 10, 20, or 50 μM. After 24 hours from the treatment with CDDP, the viability of the cells was determined by a dye-exclusion assay. The axis of ordinate represents the cell viability (%), and the axis of abscissa represents the concentration (μM) of cisplatin.

FIG. 3 shows the viability of HSP47 knockout human cancer cells treated with 5-FU. Human cancer cells (Mock) and HSP47 knockout (KO) cancer cells were treated with 5-fluorouracil (5-FU) at a concentration of 0, 1, 5, 10, 20, or 50 μM. After 24 hours from the treatment with 5-FU, the viability of the cells was determined by a dye-exclusion assay. The axis of ordinate represents the cell viability (%), and the axis of abscissa represents the concentration (μM) of 5-fluorouracil.

### DESCRIPTION OF EMBODIMENTS

In the present specification, a medicine comprising an HSP47 inhibitor for increasing the sensitivity of a cancer patient to a chemotherapeutic agent; a method and a kit using this medicine; a method for enhancing the sensitivity to a chemotherapeutic agent, the method comprising administering an HSP47 inhibitor (hereinafter, may be referred to as chemotherapeutic agent sensitivity enhancing method); and a method for treating cancer, the method comprising administering an HSP47 inhibitor and a chemotherapeutic agent (hereinafter, may be referred to as combination therapy) are provided.

The medicine, method, and kit according to the invention are characterized in using a nucleic acid molecule (for example, small interfering nucleic acid (siNA), small interfering RNA (siRNA), double-stranded RNA (dsRNA), microRNA (miRNA), or short hairpin RNA (shRNA)) that binds to transcript RNA (including pre-mRNA and mRNA) of HSP47, for example, transcript RNA of HSP47 as set forth in SEQ ID NO:1, or knocking out the HSP47 gene using a genome editing technology, for example, CRISPR-Cas9, which utilizes single-stranded guide RNA (sgRNA, gRNA) (for example, SEQ ID NO:8) specific to the HSP47 genome.

Furthermore, the medicine, method, and kit according to the invention are characterized in using a chemotherapeutic agent in combination, in addition to using the above-described nucleic acid molecule (for example, small interfering nucleic acid (siNA), small interfering RNA (siRNA), double-stranded RNA (dsRNA), microRNA (miRNA), or short hairpin RNA (shRNA)) that binds to transcript RNA of HSP47, or knocking out the HSP47 gene using a genome editing technology, for example, CRISPR-Cas9, which utilizes single-stranded guide RNA (sgRNA, gRNA) (for example, SEQ ID NO:8) specific to the HSP47 genome.

### 1. Medicine of Invention

An aspect of the invention relates to a medicine for enhancing the sensitivity of a cancer patient to a chemotherapeutic agent, the medicine comprising an HSP47 inhibitor, for example, an inhibitory agent for the expression of HSP47.

The base sequence and amino acid sequence of the HSP47 gene are known in the pertinent art, and in this invention, the mRNA sequence of HSP47 is set forth in SEQ ID NO:1.

According to the invention, the transcript RNA of HSP47 may be, for example, derived from a human, and may have a base sequence set forth in SEQ ID NO:1 or the same base sequence except that one to several nucleotides are deleted, substituted, added, or inserted, or a base sequence having a sequence identity of 70% or higher, 80% or higher, or 90% or higher, preferably 95% or higher, and more preferably 98% or higher or 99% or higher, with each of the above-described base sequences.

In the present specification, the term “several” means a number of bases of 2 to 10, preferably 2 to 5, and more preferably 2 or 3. Furthermore, the sequence identity can be determined using, for example, a known algorithm such as BLAST.

According to the invention, the inhibitory agent for the expression of HSP47 may be a component that inhibits the expression of HSP47 or a component that inhibits the functions of HSP47 protein, and examples include a siRNA having RNA interference (RNAi) action (for example, containing siHSP47-A: 5′-CUACGACGACGAGAAGGAAtt-3′ (SEQ ID NO:2), siHSP47-B: 5′-AGCCCUCUUCUGACACUAAtt-3′ (SEQ ID NO:4), or siHSP47-C: 5′-GGACAGGCCUCUACAACUAtt-3′ (SEQ ID NO:6) of the sense strand, or containing siHSP47-A: 5′-UUCCUUCUCGUCGUCGUAGta-3′ (SEQ ID NO:3), siHSP47-B: 5′-UUAGUGUCAGAAGAGGGCUgg-3′ (SEQ ID NO:5), or siHSP47-C: 5′-UAGUUGUAGAGGCCUGUCCtt-3′ (SEQ ID NO:7) of the antisense strand)) or a precursor RNA thereof (for example, shRNA), or a modified RNA thereof, or a vector containing a DNA encoding a siRNA against the transcript RNA of the HSP47 gene. Furthermore, according to the invention, the inhibitory agent for the expression of HSP47 may be, for example, a siNA, a ribozyme, a shRNA, or a miRNA. The vector may contain an antisense RNA, an antisense DNA, a DNA encoding the antisense RNA, or an antisense DNA thereof. According to the invention, it is preferable that the vector contains, for example, a sequence of 5′-CCGACTGTACGGACCCAGCTCAG-3′ (SEQ ID NO:8), and it is preferable that a vector containing the sequence of SEQ ID NO:8 further contains a Cas9 sequence (SEQ ID NO:9).

According to the invention, the siRNA may be a double-stranded RNA composed of sense RNA and antisense RNA, the double-stranded RNA having an antisense RNA containing 18 to 25 nucleotides, preferably 20 to 24 nucleotides, and more preferably 21 to 23 nucleotides, which is substantially complementary to a portion of the transcript RNA of the HSP47 gene, and having RNAi (RNA interference) action.

According to the invention, the term “complementary” means that a nucleic acid can form hydrogen bonds with another nucleic acid sequence by the classical Watson-Crick type or other non-classical type.

Furthermore, according to the invention, the term “substantially complementary” includes not only a case where all the contiguous residues of a nucleic acid sequence form hydrogen bonds with the same number of contiguous residues in another nucleic acid sequence, but also a case where, for example, 70%, 80%, and 90% of residues among all the residues of a nucleic acid sequence form hydrogen bonds with the residues of another nucleic acid sequence.

Therefore, according to the invention, the siRNA may have an antisense RNA containing a nucleotide obtained by modifying several bases of a nucleotide that is 100% complementary to a portion of the transcript RNA of the HSP47 gene.

Furthermore, according to the invention, the 3′-termini of the sense RNA and the antisense RNA may each have a protruding end of 2 to 5 nucleotides, and preferably two nucleotides. According to the invention, the siRNA may be a modified siRNA.

According to the invention, the HSP47 inhibitor may be, for example, a combination of siRNA containing a sense strand and an antisense strand, and for example, the following combinations are preferred.

Combination (siHSP47-A) of 5′-CUACGACGACGAGAAGGAAtt-3′ (SEQ ID NO:2) of the sense strand and 5′-UUCCUUCUCGUCGUCGUAGta-3′ (SEQ ID NO:3) of the antisense strand (Ambion)

Combination (siHSP47-B) of 5′-AGCCCUCUUCUGACACUAAtt-3′ (SEQ ID NO:4) of the sense strand and 5′-UUAGUGUCAGAAGAGGGCUgg-3′ (SEQ ID NO:5) of the antisense strand (Ambion)

Combination (siHSP47-C) of 5′-GGACAGGCCUCUACAACUAtt-3′ (SEQ ID NO:6) of the sense strand and 5′-UAGUUGUAGAGGCCUGUCCtt-3′ (SEQ ID NO:7) of the antisense strand (created by Nitto Denko Corporation)

Regarding the introduction of an HSP47 inhibitor into cells, examples of a known technique for introduction that can be used include, without limitations, a lipofectamine method, a lipofection method, a calcium phosphate method, an ultrasonic introduction method, an electroporation method, a particle gun method, a method of utilizing a viral vector (for example, an adenovirus vector, an adeno-associated virus vector, or a retrovirus vector), and a microinjection method.

In the case of using a viral vector, the viral titer may be 1×10³ to 1×10¹⁵p.f.u. (plaque-forming unit), and the viral vector can be used at a viral titer of preferably 1×10⁵ to 1×10¹³, more preferably 1×10⁷ to 1×10¹¹, and even more preferably 1×10⁸ to 1×10¹⁰.

The nucleic acid molecule may be used as a naked nucleic acid or may be used after being incorporated into various nucleic acid constructs or vectors. Regarding the vectors, any known vectors such as a plasmid vector, a phage vector, a phagemid vector, a cosmid vector, and a viral vector can be utilized. It is preferable that the nucleic acid constructs or vectors include at least appropriate transcriptional or translational regulatory sequences derived from, for example, mammalian, microbial, viral, or insect genes. Such regulatory sequences include a sequence having a regulatory role for gene expression, for example, a transcription promoter or enhancer, an operator sequence for regulating transcription, a sequence encoding a ribosome binding site within messenger RNA, and a sequence appropriate for regulating transcription, translation initiation, or transcription termination.

Since the medicine according to the invention includes the above-described HSP47 inhibitor, the medicine can enhance the sensitivity of a cancer patient to a chemotherapeutic agent and is useful as an active ingredient for a medicine. The medicine according to the invention may include a chemotherapeutic agent in addition to an HSP47 inhibitor. The medicine according to the invention may include the above-described HSP47 inhibitor and one or two or more of pharmaceutically acceptable surfactants, carriers, diluents, and/or excipients. The pharmaceutically acceptable carriers, diluents, and the like are well known in the medical field and are described in, for example, Remington's Pharmaceutical Sciences, 18^{th} Ed., Mack Publishing Co., Easton, Pa. (1990), the entire disclosure of which is incorporated herein by reference.

The medicine can be used for, for example, an enhancement in sensitivity to a chemotherapeutic agent, and more particularly, cancer treatment carried out concomitantly with an enhancement in sensitivity to a chemotherapeutic agent, depending on the constituent elements of the medicine.

Whether the sensitivity to a chemotherapeutic agent has increased when an HSP47 inhibitor is used in combination, is determined by considering whether a 50% viability can be induced by a lower concentration of the chemotherapeutic agent when this concentration is compared with the concentration of the chemotherapeutic agent required to induce a cancer cell viability of 50% without using the HSP47 inhibitor. When a relative value of the concentration of the chemotherapeutic agent required to induce a cancer cell viability of 50% without using the HSP47 inhibitor is designated as 100, in a case where the relative value of the chemotherapeutic agent concentration is less than 100, it can be said that sensitivity has increased, and in a case where the relative value is less than 50, it can be said that sensitivity has markedly increased. When the relative value of the chemotherapeutic agent concentration is less than 50, anticancer treatment can be carried out while reducing the risk of side effects caused by the chemotherapeutic agent.

The cancer cell viability represents a relative value (%) of the number of surviving cancer cells when the number of cancer cells before treatment with an HSP47 inhibitor or a chemotherapeutic agent is designated as 100%, and can be measured by a Dye-exclusion assay. The Dye-exclusion assay enables measurement of the number of surviving cells by, for example, determining the life or death of a cell on the basis of the presence or absence of trypan blue staining. The dye-exclusion method based on trypan blue staining can be carried out by, for example, mixing equal amounts of a 0.4% trypan blue solution (selling agency: FUJIFILM Wako Pure Chemical Corporation, product code: 20717081) and a cell suspension obtained by suspending cells in a culture medium, and counting the number of stained cells and unstained cells on a hemocytometer.

The cancer for which the invention is intended is not limited so long as the cancer expresses transcript RNA of HSP47, and examples include sarcomas such as fibrosarcoma, malignant fibrous histiocytoma, liposarcoma, rhabdomyosarcoma, leiomyosarcoma, angiosarcoma, Kaposi's sarcoma, lymphangiosarcoma, synovial sarcoma, chondrosarcoma, and osteosarcoma; carcinomas such as brain tumor, head and neck cancer, breast cancer, lung cancer, esophageal cancer, stomach cancer, duodenal cancer, appendiceal cancer, colorectal cancer, rectal cancer, colon cancer, liver cancer, pancreatic cancer, gallbladder cancer, bile duct cancer, anal cancer, kidney cancer, ureteral cancer, urinary bladder cancer, prostate cancer, penile cancer, testicular cancer, uterine cancer, ovarian cancer, vulvar cancer, vaginal cancer, and skin cancer; leukemia, and malignant lymphoma. Among these, solid tumors are preferable, and breast cancer, colorectal cancer, colon cancer, lung cancer, and pancreatic cancer are more preferable.

Whether cancer cells express HSP47 can be determined by detecting the expression of transcript RNA of HSP47 at the gene level. Specifically, at the gene level, for example, detection can be made by any known gene expression analysis method such as a Northern blotting method, an RNase protection assay, PCR methods such as RT-PCR and real-time PCR, an in situ hybridization method, or an in vitro transcription method, and detection by real-time PCR is preferable. Furthermore, whether cancer cells express HSP47 can be determined by detecting the expression of HSP47 at the protein level. Specifically, for example, an immunoprecipitation method, EIA (enzyme immunoassay) (for example, ELISA (enzyme-linked immunosorbent assay)), RIA (radioimmunoassay) (for example, IRMA (Immunoradiometric assay), RAST (radioallergosorbent test), and RIST (radioimmunosorbent test)), a Western blotting method, an immunohistochemistry method, an immunocytochemistry method, or a flow cytometry method can be used for detection. The transcript RNA of HSP47 is expressed in cancer cells; however, generally, the transcript RNA of HSP47 is substantially not expressed in normal cells, except for fibroblasts and myofibroblasts. According to the invention, cancer cells express HSP47 protein.

According to an embodiment of the invention, the medicine of the invention includes an HSP47 inhibitor for increasing the sensitivity of a cancer patient to a chemotherapeutic agent. According to another embodiment of the invention, the medicine of the invention may further include another chemotherapeutic agent that is useful for treating a target disease.

Regarding the chemotherapeutic agent, a chemotherapeutic agent used for cancer treatment, such as a cell-permeable anticancer agent, is preferable, and as the cell-permeable anticancer agent, without limitations, a chemotherapeutic agent having cell damaging action such as cytocidal action and proliferation inhibitory action, for example, an alkylating agent, an antimetabolite (for example, 5-fluorouracil or gemcitabine), an antitumor antibiotic substance, an alkaloid, a hormonal therapeutic agent, a platinum complex (for example, cisplatin), an angiogenesis inhibitory agent, a topoisomerase inhibitory agent, and a microtubule agonist may be included. Among these, an alkylating agent, an antimetabolite, an antitumor antibiotic substance, a platinum complex, or a topoisomerase is a chemotherapeutic agent capable of inducing DNA synthesis inhibition and DNA damage and inducing endoplasmic reticulum stress, and is preferable from the viewpoint that when the chemotherapeutic agent is used in combination of an HSP47 inhibitor, the anticancer action by the chemotherapeutic agent is particularly enhanced. For the medicine according to the invention, a combination of an HSP47 inhibitor and cisplatin or 5-fluorouracil is most preferred. In the case of administering these in combination, a preferable concentration of the HSP47 inhibitor is 1 nM to 100 nM as the final concentration, and a preferable concentration of cisplatin and 5-fluorouracil is 5 μM to 100 μM.

In a case where an HSP47 inhibitor and a chemotherapeutic agent are used in combination for the medicine according to the invention, these may be included in a single composition or may be included separately in a plurality of compositions, and those compositions may be separately used or may be used in combination.

According to various embodiments of the invention, the above-described medicine can be used in a state of being carried on various drug delivery carriers. Such carriers are not limited, and examples include polymer nanoparticles, polymicelles, dendrimers, liposomes, virus nanoparticles, and carbon nanotubes (see Cho K. et al., Clin Cancer Res. 2008 Mar. 1; 14(5):1310-6, and the like).

The medicine according to the invention may be administered by various routes including both oral and parenteral routes, examples of which include, without limitations, routes such as oral, intravenous, intramuscular, subcutaneous, topical, rectal, intratumoral, intraarterial, intraportal, intramedullary, intrapulpal, sublingual, intraoral, intraventricular, transmucosal, transdermal, intranasal, intraperitoneal, intrapulmonary, and intrauterine routes, and the medicine may be formulated into a dosage form appropriate for each of the administration routes. Regarding such a dosage form and preparation method, any known dosage form and method can be appropriately employed (see, for example, Standard Pharmaceutics, edited by Watanabe Yoshiteru, et al., Nankodo, 2003; and Remington's Pharmaceutical Sciences).

Examples of a dosage form appropriate for oral administration include, without limitations, a powder, a granular preparation, a tablet, a capsule, a liquid preparation, a suspension, an emulsion, a gel preparation, and a syrup preparation, and examples of a dosage form appropriate for parenteral administration include injectable preparations such as a solution injectable preparation, a suspension injectable preparation, an emulsion injectable preparation, and a prepared-upon-use type injectable preparation. The preparation for parenteral administration can be in the form of an aqueous or non-aqueous isotonic sterile solution or suspension.

### 2. Kit of Invention

The invention also relates to a preparation kit of compositions comprising one or two or more containers that include an active ingredient (for example, HSP47 inhibitor) that can be included in the medicine according to the invention and a chemotherapeutic agent as necessary, singly or in combination; and necessary constituent elements of a medicine provided in the form of such a kit. The kit according to the invention may include, in addition to those described above, instructions in which a preparation method, administration method, and the like of the medicine according to the invention, for example, an instruction manual or an electronic recording medium such as a CD or a DVD.

### 3. Method of Invention

An aspect of the invention relates to a method for increasing the sensitivity of a cancer patient to a chemotherapeutic agent, the method comprising administering an HSP47 inhibitor (chemotherapeutic agent sensitivity enhancing method).

Another aspect of the invention relates to a method for treating cancer, the method comprising administering an HSP47 inhibitor and a chemotherapeutic agent (combination therapy).

With regard to the methods according to the invention, the patient who receives administration is typically a cancer patient in need of treatment by means of a chemotherapeutic agent. Examples of such a patient include, without limitations, patients who are suffering from the above-described cancers related to high expression of HSP47, who have been diagnosed to have these cancers, or who have a high risk of developing these cancers. Therefore, the methods according to the invention may further include a step of identifying a patient in need of treatment using the medicine according to the invention, the medicine comprising an HSP47 inhibitor. The above-described step is, for example, a step of quantitatively determining the expression of mRNA or protein of HSP47 in a tumor tissue isolated from a cancer patient. Specific examples of the cancer are as described above in relation to the medicine.

With regard to the methods according to the invention, the medicine according to the invention can be used in combination with another active substance or treatment method useful for treating the target cancer. The methods according to the invention can be used in combination with physical therapy such as radiation treatment, surgical treatment such as surgical operation, or the like as the treatment method. In the case of treating a cancer patient by surgical treatment, the methods can be applied to either or both of the preoperative chemotherapy and the postoperative chemotherapy of the invention.

An effective amount in the therapeutic or treatment method according to the invention is, for example, an amount with which symptoms of a disease are reduced, or the progression of the disease is delayed or stopped, and preferably, the effective amount is an amount with which a disease is suppressed or cured. Furthermore, an amount that does not cause adverse effects exceeding the benefit of administration is preferable. Such an amount can be appropriately determined by an in vitro test using cultured cells or the like, or a test with a model animal such as a mouse, a rat, a dog, or a pig, and such testing methods are well known to those ordinarily skilled in the art. Furthermore, the dosage of the drug that is used for the treatment method according to the invention is known to those ordinarily skilled in the art or can be appropriately determined by the above-described tests or the like. According to the invention, a preferable concentration of a chemotherapeutic agent such as cisplatin and 5-fluorouracil is 5 μM to 100 μM.

Specific dosage of the active ingredient (for example, HSP47 inhibitor) that is administered in the methods according to the invention as described in the present specification can be determined by considering various conditions related to the subject who requires treatment, for example, severity of the symptoms, general health condition of the subject, age, body weight, gender of the subject, diet, timing and frequency of administration, medicine that is being used in combination, responsiveness to therapy, dosage form, and compliance to therapy. The dosage of the drug used for the methods according to the invention is preferably, in the case of human being, for example, about 0.1 mg to about 1,000 mg per dose, in terms of siRNA molecules per kg of body weight for an adult. The concentration of the drug used for the treatment method according to the invention is preferably, in the case of siRNA, preferably 5 nM to 180 μM.

Regarding the route of administration, various routes including both oral and parenteral routes, for example, routes such as oral, intravenous, intramuscular, subcutaneous, topical, intratumoral, rectal, intraarterial, intraportal, intramedullary, intrapulpal, sublingual, intraoral, intraventricular, transmucosal, transdermal, intranasal, intraperitoneal, intrapulmonary, and intrauterine routes are included.

The frequency of administration varies depending on the properties of the agent or composition used, or the conditions of the subject including the above-described items; however, for example, the frequency of administration may be several times a day (that is, 2, 3, 4 times, or 5 or more times, a day), once a day, once in every several days (that is, every 2, 3, 4, 5, 6, or 7 days), once in every week, or once in several weeks (that is, once in 2, 3, or 4 weeks).

In the combination therapy according to the invention, an HSP47 inhibitor and a chemotherapeutic agent may be administered separately or may be administered simultaneously; however, in the case of administering the agents separately, it is preferable to administer the HSP47 inhibitor first. In a case where the chemotherapeutic agent is administered after the administration of the HSP47 inhibitor, the administration of the chemotherapeutic agent is preferably after 1 hour to after 70 hours, more preferably after 2 hours to after 60 hours, and particularly preferably after 4 hours to after 50 hours, from the administration of the HSP47 inhibitor. When the chemotherapeutic agent is administered after 4 hours to after 50 hours from the administration of the HSP47 inhibitor, cancer treatment by the chemotherapeutic agent can be achieved in a state in which decrease in the expression of HSP47 protein has been sufficiently exhibited, and therefore, the viability of cancer cells can be markedly decreased.

As used in the present specification, the term “patient” means any biological individual; however, the patient is, for example, an animal, a mammal, or a human individual. According to the invention, the term “patient” typically means a cancer patient who requires treatment with a chemotherapeutic agent.

Furthermore, the term “treatment” as used in the present specification is meant to include all kinds of medically acceptable prophylactic and/or therapeutic interventions intended for curing, transient remission or prevention of a disease, or the like. For example, the term “treatment” includes medically acceptable interventions for a variety of purposes, including delaying or stopping of the progression of a disease, involution or elimination of lesions, prevention of onset, prevention of recurrence, or the like.

This time, it has been disclosed by the inventors that as the medicine according to the invention suppresses the expression of transcript RNA of HSP47 (SEQ ID NO:1), the sensitivity to a chemotherapeutic agent can be enhanced in a cell line of breast cancer, colorectal cancer, colon cancer, or pancreatic cancer, and consequently, combination therapy for cancer is enabled.

Furthermore, as a result of the invention, the amount of administration of a chemotherapeutic agent can be reduced to a large extent in cancer treatment, and anticancer treatment can be carried out while reducing the risk of side effects caused by the chemotherapeutic agent.

Therefore, the invention also relates to a medicine for providing the above-described action, the medicine comprising an HSP47 inhibitor; use of an HSP inhibitor in the production of a medicine for providing the above-described action; and use of an HSP47 inhibitor for providing the above-described action.

### EXAMPLES

The invention will be described more specifically by way of the following Examples; however, the technical scope of the invention is not intended to be limited to these Examples.

### Example 1: Acquisition and Culture of Cell Lines

All the human cancer cell lines (MDA-MB-231, HCT116, SW480, PANC-1, Suit2, and MIA-PaCa-2) used in the present specification were purchased from American Type Culture Collection. MDA-MB-231 cells, HCT116 cells, SW480 cells, Suit2 cells, and MIA-PaCa-2 cells were cultured in Dulbecco's Modified Eagle Medium (DMEM, Sigma-Aldrich, St. Louis, Mo.) supplemented with 10% fetal bovine serum (FBS, Invitrogen Life Technologies). PANC-1 cells were cultured in RPMI1640 medium supplemented with 10% FBS.

### Example 2: Transfection with siRNA

Cancer cells were transfected with control siRNA (siControl, Ambion, Foster City, Calif.), HSP47-AsiRNA (siHSP47-A, sense: 5′-CUACGACGACGAGAAGGAAtt-3′ (SEQ ID NO:2); antisense: 5′-UUCCUUCUCGUCGUCGUAGta-3′ (SEQ ID NO:3)), HSP47-B siRNA (siHSP47-B, sense: 5′-AGCCCUCUUCUGACACUAAtt-3′ (SEQ ID NO:4); antisense: 5′-UUAGUGUCAGAAGAGGGCUgg-3′ (SEQ ID NO:5)), or HSP47-C siRNA (siHSP47-C, sense: 5′-GGACAGGCCUCUACAACUAtt-3′ (SEQ ID NO:6); antisense: 5′-UAGUUGUAGAGGCCUGUCCtt-3′ (SEQ ID NO:7)), using Lipofectamine RNAiMAX (Invitrogen Life Technologies), and the cancer cells were cultured for 5 hours at 37° C. in the air. Five hours after the transfection with siRNA, the cells were cultured in DMEM supplemented with 10% FBS.

### Example 3: Establishment of HSP47^{-/-} Cells

pCG SapI vector (acquired from Dr. Sakurai, Graduate School of Medicine, Shinshu University) includes cloning sites for inserting a Cas9 sequence (SEQ ID NO:9) and a target gRNA sequence (SEQ ID NO:8) (Uemura et al., Sci Rep. 2016 Oct. 26; 35861, Takei et al., Sci. Rep. 2017 Aug. 24 7(1); 9389). A gRNA sequence (5′-CCGACTGTACGGACCCAGCTCAG-3′ (SEQ ID NO:8)) specifically designed for exon 2 of the human HSP47 genome was inserted into the pCG SapI vector. The targeting vector thus constructed and pcDNA3.1(+) were co-transfected into cancer cells (SW480, HCT116, PANC1, Suit2, MIA-PaCa-2, and MDA-MB-231) using Lipofectamine 2000 (Thermo Fisher Scientific). The cells were cultured in DMEM medium supplemented with 10% FBS and G418 (1000 μg/mL), and cell lines that stably silenced HSP47 were selected. Single cell clones were evaluated by a DNA sequencing analysis, and indels (insertions and deletions) in target alleles were detected. Furthermore, the expression level of HSP47 protein in the selected clones was checked by Western blotting. Control cell clones (mock) were created by transfecting an empty vector.

### Example 4: Viability of Cells Treated with Anticancer Agent

The cancer cells transfected in Examples 2 and 3 were treated for 24 hours with cis-diaminedichloroplatinum (II) (CDDP, Sigma-Aldrich) at a concentration of 1, 5, 10, 20, or 50 μM, or with 5-fluorouracil (Wako Pure Chemical, Tokyo, Japan) at a concentration of 1, 5, 10, 20, or 50 μM. The viability of the treated cells was determined by a Dye-exclusion assay.

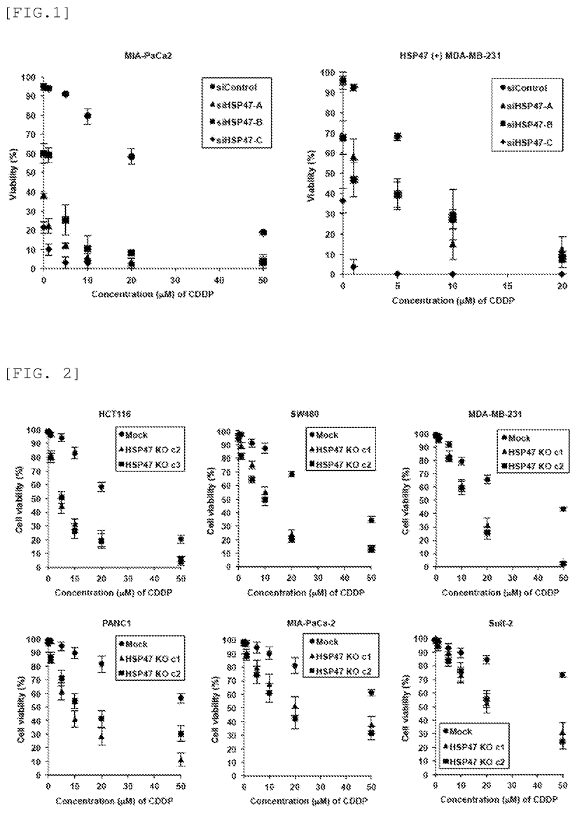

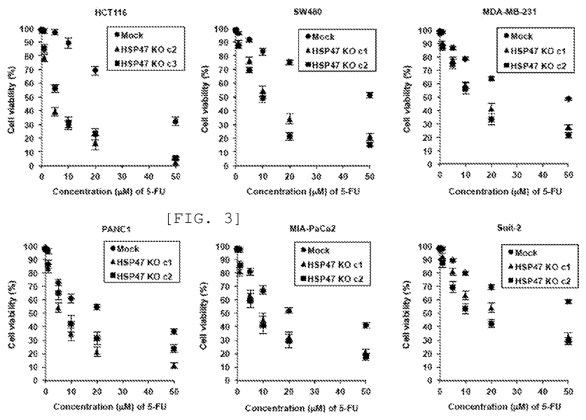

The results are shown in FIGS. 1 to 3. All data are presented as mean±standard deviation. Differences between groups were tested for statistical significance using Student's t-test or analysis of variance (ANOVA). Statistical significance was determined as P<0.05.

It was found that the medicine according to the invention enhances the sensitivity of various cancer cells such as breast cancer, colorectal cancer, colon cancer, pancreatic adenocarcinoma, or pancreatic cancer to chemotherapeutic agents, by inhibiting the expression of HSP47.

In FIG. 1, when the concentration relative value of a chemotherapeutic agent required to induce a viability of cancer cells of 50% without using an HSP47 inhibitor (siControl group) was designated as 100, in a case where HSP47 inhibitors were used (siHSP47-A, siHSP47-B, and siHSP47-C), the relative values of chemotherapy were all less than 50, and sensitivity was markedly increased.

1. (canceled)

2. The method according to claim 10, wherein the HSP47 inhibitor is an interfering nucleic acid against HSP47, a ribozyme, an antisense nucleic acid, a microRNA, a short hairpin RNA, a vector expressing any of these, or a cell transformed with any of these.

3. The method according to claim 2, wherein the interfering nucleic acid against HSP47 is a siNA or a siRNA.

4. The method according to claim 10, wherein the cancer patient is a patient with breast cancer, colorectal cancer, colon cancer, or pancreatic cancer.

5. The method according to claim 10, wherein the chemotherapeutic agent is an alkylating agent, an antimetabolite, an antitumor antibiotic substance, an alkaloid, a hormonal therapeutic agent, a platinum complex, an angiogenesis inhibitory agent, a topoisomerase inhibitory agent, or a microtubule agonist.

6. The method according to claim 10, wherein the chemotherapeutic agent is cisplatin (CDDP) or 5-fluorouracil (5-FU).

7-9. (canceled)

10. A method for increasing sensitivity of a cancer patient to a chemotherapeutic agent, comprising administering an effective amount of an HSP47 inhibitor to a cancer patient.

11. The method according to claim 10, further comprising a step of identifying a patient in need of treatment using a medicine comprising an HSP47 inhibitor.

12. The method according to claim 11, wherein the step is a step of quantitatively determining the HSP47 expression in a tumor tissue isolated from a cancer patient.

13. A method for treating cancer, comprising administering effective amounts of an HSP47 inhibitor and a chemotherapeutic agent to a cancer patient.

14. (canceled)
